(19) United States
(12) Patent Application Publication
(10) Pub. No.: US 2022/0009874 A1
(43) Pub. Date: Jan. 13, 2022

(54) CATALYTIC SYNTHESIS OF ANTI-UV AND ANTIOXIDANT CONJUGATED 8-8 DIMERS IN A GREEN SOLVENT

(71) Applicant: INSTITUT DES SCIENCES ET INDUSTRIES DU VIVANT ET DE L'ENVIRONNEMENT - AGROPARIS TECH, Paris Cedex 05 (FR)

(72) Inventors: Florent Allais, Bouy (FR); Matthieu Mention, Betheny (FR); Amandine Flourat, La Neuvillette, Reims (FR)

(21) Appl. No.: 17/288,796

(22) PCT Filed: Oct. 25, 2019
(86) PCT No.: PCT/FR2019/052547

§ 371 Date: Apr. 26, 2021

(30) Foreign Application Priority Data
Oct. 26, 2018 (FR) 1859902

(51) Int. Cl. C07C 69/736; B01J 31/18; C07C 67/465

(52) U.S. Cl. CPC C07C 69/736; B01J 31/181; B01J 2231/641; B01J 2531/16; C07C 67/465

(57) ABSTRACT

A process for preparing b-b dimers having anti-UV and antioxidant properties, from p-hydroxycinnamic esters and amides disubstituted in the ortho position with respect to the phenol function and from ketones disubstituted in the ortho position with respect to the phenol, in particular, from sinapic acid esters and amides and ketone analogs. The dimers of formulae (I), (II), (III) and (IV) as obtained by means of the process according to the present disclosure can be used for the production of polymers/plastics (in plastics technology), or for the protection of plants against the cold and as cosmetic or food-processing ingredients, for example. The process may be used to form biobased anti-UV molecules.

### CROSS-REFERENCE TO RELATED APPLICATIONS

This application is a national phase entry under 35 U.S.C. § 371 of International Patent Application PCT/FR2019/052547, filed Oct. 25, 2019, designating the United States of America and published as International Patent Publication WO 2020/084266 A1 on Apr. 30, 2020, which claims the benefit under Article 8 of the Patent Cooperation Treaty to French Patent Application Serial No. 1859902, filed Oct. 26, 2018.

### TECHNICAL FIELD

The present disclosure relates to biobased and antioxidant anti-UV molecules. In particular, the present disclosure relates to a novel process for preparing β-β dimers (also referred to as 8-8) having anti-UV and antioxidant properties, from p-hydroxycinnamic esters, amides and ketones disubstituted in the ortho position with respect to the phenol, in particular, from sinapic acid esters or amides and ketone analogs. The dimers of formulae (I), (II), (III) and (IV) as obtained by means of the process according to the present disclosure can be used for the production of polymers/plastics (in plastics technology), for the protection of plants against the cold and as a cosmetic or food-processing ingredient.

### BACKGROUND

The use of agroresources is a major issue for replacing compounds derived from fossil resources. Among the molecules of interest are anti-UV and antioxidant molecules (in the context of the present disclosure, “antioxidant” is intended to mean “antioxidant and/or anti-free radical”). It is known, for example, that sinapic acid, and some of its esters, absorb in the UV range. Thus, from sinapic acid, present predominantly in the Brassicaceae, it is possible to access molecules that can be used as anti-UV and/or antioxidant.

Processes making it possible to obtain sinapic acid derivatives of “dimer” type are described in the prior art:

Bunzel, M et al. describe a reaction carried out from sinapic ester and manganese salts; however, large-scale reproducibility cannot be achieved (J. Agric. Food Chem. 2003, 51, 1427-1434).

Neudorffer, A et al. describe a reaction involving a sinapic ester and tetraethylammonium perchlorate (explosive product) with tetramethylammonium hydroxide (corrosive product) in acetonitrile (expensive product) (J. Agric. Food Chem. 2004, 52, 2084-2091); this process is not viable on a large scale, either from a safety point of view or from an environmental point of view.

Patent JP2011/195465 describes a process for producing multimers from sinapic esters and iron chloride (involving a ferulic acid).

Finally, Sathish Kumar, B. et al. describe a preparation process involving syringaldehyde derivatives and via a double aldol reaction/crotonation (Bioorg. Med. Chem. 2014, 22, 1342-1354); this process does not make it possible to directly obtain molecules bearing free phenols.

### BRIEF SUMMARY

A novel ecological and industrializable process is proposed for the preparation of dimeric molecules having anti-UV and/or antioxidant properties, derived from esters or amides of p-hydroxycinnamic acids disubstituted in the ortho position with respect to the phenol function (such as sinapic acid) or ketone analogues. This process paves the way for novel applications of these molecules in many fields such as plastics technology, agronomy, cosmetics and the field of fragrances.

The present disclosure therefore relates to a novel route for the synthesis of β-β dimers using an amine as solvent and reagent. In a more ecological variant, part of the amine is advantageously replaced by a polar aprotic solvent, the amine no longer being used other than as a reagent in a catalytic amount. Among the polar aprotic solvents tested, dihydrolevoglucosenone (2H-LGO, CYRENE®), a biobased green solvent derived from cellulose, made it possible to significantly increase the yield while reducing the reaction time. In addition, using 2H-LGO as solvent makes it possible to dispense with the purification step, which simplifies the process.

### BRIEF DESCRIPTION OF THE DRAWINGS

The present disclosure is illustrated with the aid of the following examples.

FIG. 1: UV spectrum of ethyl β-β disinapate (A) compared to that of sinapoyl malate (B);

FIG. 2: UV spectrum of methylated ethyl β-β disinapate (A) compared to that of sinapoyl malate (B);

FIG. 3: UV spectrum of acetylated ethyl β-β disinapate (A) compared to that of sinapoyl malate (B);

FIG. 4: UV spectrum of ethyl β-β didihydrosinapate (A) compared to that of sinapoyl malate (B);

FIG. 5: UV spectrum of β-β disinapilic alcohol (A) compared to that of sinapoyl malate (B);

FIG. 6: UV spectrum of tert-butyl β-β disinapate (A) compared to that of sinapoyl malate (B);

FIG. 7: UV spectrum of di-tert-butyl malate β-β disinapate (A) compared to that of sinapoyl malate (B);

FIG. 8: UV spectrum of heptyl β-β disinapate (A) compared to that of sinapoyl malate (B);

FIG. 9: UV spectrum of 2-ethylhexyl β-β disinapate (A) compared to that of sinapoyl malate (B);

FIG. 10: UV spectrum of guaiacol β-β disinapate (A) compared to that of sinapoyl malate (B);

FIG. 11: UV spectrum of ethyl β-β diferulate (A) compared to that of sinapoyl malate (B);

FIG. 12: UV spectrum of isopropyl β-β disinapate (A) compared to that of sinapoyl malate (B);

FIG. 13: UV spectrum of malic acid β-β disinapate (A) compared to that of sinapoyl malate (B);

FIG. 14: UV spectrum of β-β disinapilic acetone (A) compared to that of sinapoyl malate (B);

FIG. 15: UV spectrum of N-phenylamide β-β disinapate (A) compared to that of sinapoyl malate (B);

FIG. 16: UV spectrum of ethyl β-β disinapate (A) and of compounds II and III derived therefrom, respectively, ethyl β-β dihydrosinapate (C) and the β-β dimer of disinapilic alcohol (B);

FIG. 17: Antioxidant activity of the β-β dimeric compounds; and

FIG. 18: Antioxidant activity of the β-β dimeric compounds.

### DETAILED DESCRIPTION

A first object of the present disclosure relates to a process for synthesizing a β-β dimer of formula (I):

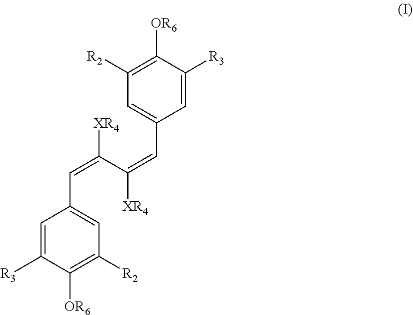

wherein:
   R₂ and R₃ are independently selected from a linear, cyclic or branched C₁ to C₈ alkyl group or a linear, cyclic or branched C₁ to C₈ O-alkyl group;
   R₄ is an H, a linear, cyclic or branched C₁ to C₃₀ alkyl group, or an organic (mono- or di-)acid hydro group;
   R₆ is an H, an acetyl group or a linear, cyclic or branched C₁ to C₈ alkyl group, a silyl, benzyl or benzoyl protecting group;
   X is a C═O group, a CO₂ group or a C(O)NR₅ group, wherein R₅ is an H or a linear, cyclic or branched C₁ to C₈ alkyl group;
from esters or amides of a para-hydroxycinnamic acid disubstituted in the ortho position with respect to the phenol function (such as sinapic acid) when X is a CO₂ or C(O)NR₅ group, or from ketones of formula (C):

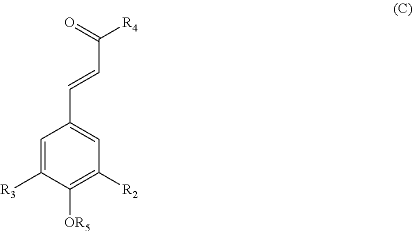

wherein:
   R₂ and R₃ are independently selected from a linear, cyclic or branched C₁ to C₈ alkyl group or a linear, cyclic or branched C₁ to C₈ O-alkyl group;
   R₄ is an H, a linear, cyclic or branched C₁ to C₃₀ alkyl group, or an organic (mono- or di-)acid hydro group;
   R₆ is an H, an acetyl group or a linear, cyclic or branched C₁ to C₈ alkyl group, a silyl, benzyl or benzoyl protecting group;
when X is a C═O group,
wherein this process implements a catalytic reaction with copper in the presence of an amine, alone or in combination with a polar aprotic solvent.

The amine involved in this process can be chosen from pyridine, 4-dimethylaminopyridine (DMAP), piperidine, piperazine, trimethylamine, pyrrolidine and aniline. In a preferred embodiment, the amine is an aromatic amine chosen from pyridine and DMAP.

When the amine is used alone, it acts both as solvent and reagent as a catalyst for the reaction.

Among the dimers of formulae (I) obtained by the process according to the present disclosure, mention may be made of ethyl β-β disinapate, heptyl β-β disinapate, tert-butyl β-β disinapate, isopropyl β-β disinapate 2-ethylhexyl β-β disinapate, di-tert-butyl malate β-β disinapate, cresol β-β disinapate, guaiacol β-β disinapate, reduced ethyl β-β disinapate, β-β disinapyl alcohol, kojate β-β disinapate, β-β disinapyl acetone, N-phenylamide β-β disinapate and malic acid β-β disinapate.

With a view to making the process more ecological, a large part of the amine has been replaced, which has a certain toxicity, with a polar aprotic solvent. In this embodiment, the amount of amine can be reduced until it acts only as a reagent where it is used in a catalytic amount.

Among the polar aprotic solvents that can be used in this process, mention may be made of N-methyl-2-pyrrolidone (NMP), dimethylformamide (DMF), dimethyl sulfoxide (DMSO), 1,4-dioxane, tetrahydrofuran (THF), dichloromethane (DCM) and dihydrolevoglucosenone (2H-LGO).

Thus, in a preferred embodiment, the amine, in particular, the pyridine, is partially replaced by DMSO. This substitution may correspond to decreasing the amine at least by a factor of 2, or even at least by a factor of 5, preferably at least by a factor of 10, at least by a factor of 20 and most particularly preferably at least by a factor of 30. Experience shows that this substitution can be carried out until the amount of pyridine decreases by a factor of 34. Not only is the amount of amine reduced, but the yield and selectivity of the reaction are maintained while reducing the reaction time (from 24 to 20 hours). Thus, the reaction can be carried out with an amount of amine (for example, pyridine) that is stoichiometric with respect to that of the substrate (for example, ethyl sinapate).

In another preferred embodiment, the amine, in particular, the pyridine, is partially replaced by 2H-LGO, a particularly beneficial green solvent. This substitution may correspond to decreasing the amine at least by a factor of 2, or even at least by a factor of 5, preferably at least by a factor of 10, at least by a factor of 20 or even at least by a factor of 30, most particularly preferably at least by a factor of 40. Experience shows that this substitution can be carried out until the amount of pyridine decreases by a factor of 45. Compared to the use of DMSO, not only is the amount of amine reduced even further, but the yield and selectivity of the reaction are significantly improved (from 61% to 89% or 90%) while reducing the reaction time (from 20 to 7 hours). Thus, the process using an amine and 2H-LGO has many advantages: the amount of amine is reduced as far as possible, resulting in limited toxicity, the reaction is highly selective, the yield is high (89%), the reaction time is reduced (7 hours), the solvent used is biobased and the dimer is isolated after a simple liquid/liquid extraction without a purification step. This is an industrializable and ecological process for synthesizing compounds with anti-UV and antioxidant properties.

In a preferred embodiment, the process uses pyridine as catalytic reagent and 2H-LGO as solvent. The use of 2H-LGO as solvent is particularly advantageous; indeed, since the conversion and the yield are better, the process for obtaining the dimers of interest can be implemented without a purification step.

Thus, the process according to the present disclosure is a greener and more sustainable alternative, but one that is also more reproducible on a large scale than the processes currently proposed.

In a particular embodiment, the present disclosure relates to a process for synthesizing a β-β dimer of formula (I′):

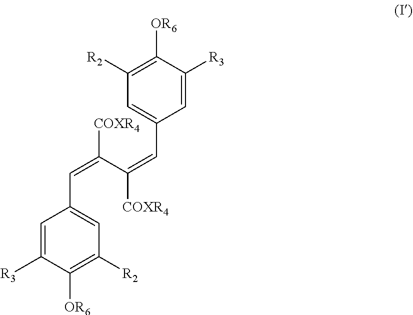

wherein:
   R₂ and R₃ are independently selected from a linear, cyclic or branched C₁ to C₈ alkyl group or a linear, cyclic or branched C₁ to C₈ O-alkyl group;
   R₄ is a linear, cyclic or branched C₁ to C₃₀ alkyl group or an organic (mono- or di-)acid hydro group;
   R₆ is an H, an acetyl group or a linear, cyclic or branched C₁ to C₈ alkyl group, a silyl, benzyl or benzoyl protecting group;
   X is an O, an NR₅ group wherein R₅ is an H or a linear, cyclic or branched C₁ to C₈ alkyl group;
from esters or amides of a para-hydroxycinnamic acid disubstituted in the ortho position with respect to the phenol function (such as sinapic acid), wherein this process implements a catalytic reaction with copper in the presence of an amine, alone or in combination with a polar aprotic solvent.

The compound of formula (I′) can be substituted for the compound of formula (I) in the remainder of the text.

The compound of formula (I) or of formula (I′) can be converted into a compound of formula (II) or (III) or (IV) using, respectively, reduction of the unsaturation, reduction of the carbonyl compound (COXR₄), or a combination of both. The person skilled in the art will easily determine the possible conversion of the R₄ and R₆ groups under these reduction conditions.

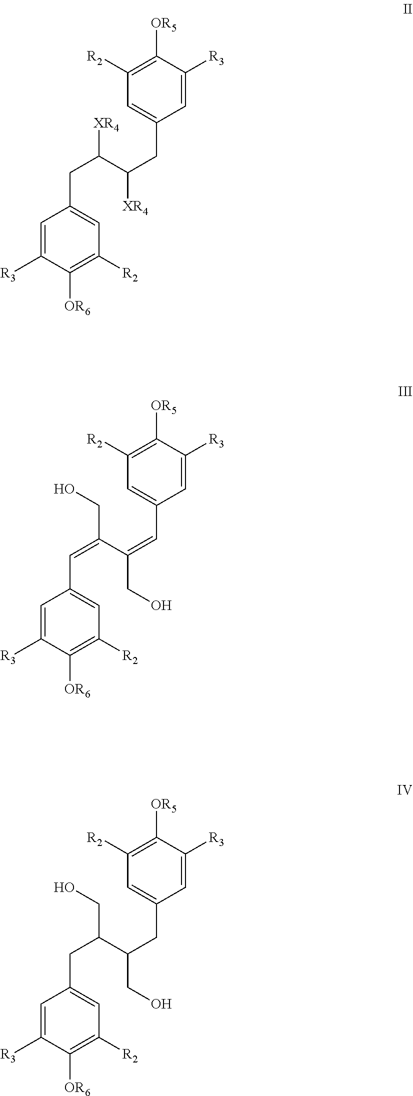

In a particular embodiment of the present disclosure, the compound of formula (I) can be converted into a compound of formula (II′) by applying the same reduction reactions:

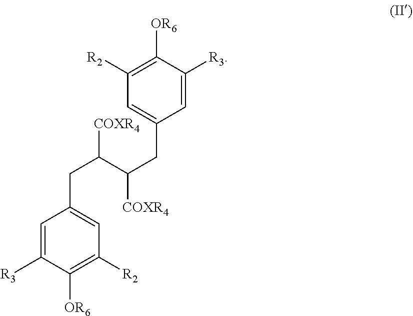

Thus, in a particular embodiment, the present disclosure relates to a process for preparing a dimer of formula (II), (III) or (IV) obtained by reduction of a dimer of formula (I) or of formula (I′) as defined above. In other words, the process for synthesizing a β-β dimer of formula (I) or of formula (I′) can comprise an additional step of reduction(s) to synthesize the dimers of formula (II), (III) or (IV).

In a particular embodiment, the dimer of formula (I) is the dimer of ethyl β-β sinapate (ethyl β-β disinapate), wherein:
R₂=R₃=OMe,
X═CO₂,
R₄=Et, and
R₆=H,
the corresponding compounds of formulae (II) and (III) and (IV) then being, respectively, ethyl β-β didihydrodisinapate, the β-β dimer of disinapyl alcohol and the β-β dimer of didihydrosinapyl alcohol (2,3-bis(4-hydroxy-3,5-dimethoxybenzyl)butane-1,4-diol).

The compound of formula (I) can also be the β-β dimer of sinapoyl malate (or β-β disinapoyl malate), wherein:
R₂=R₃=OMe,
X═CO₂,
R₄=CH(CO₂H)(CH₂CO₂H), and
R₆=H.

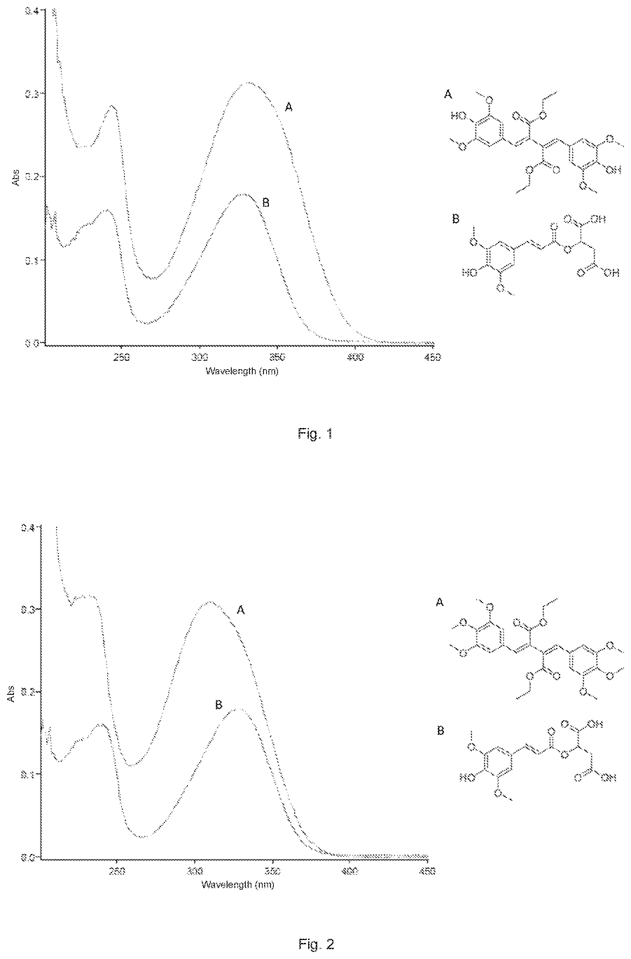

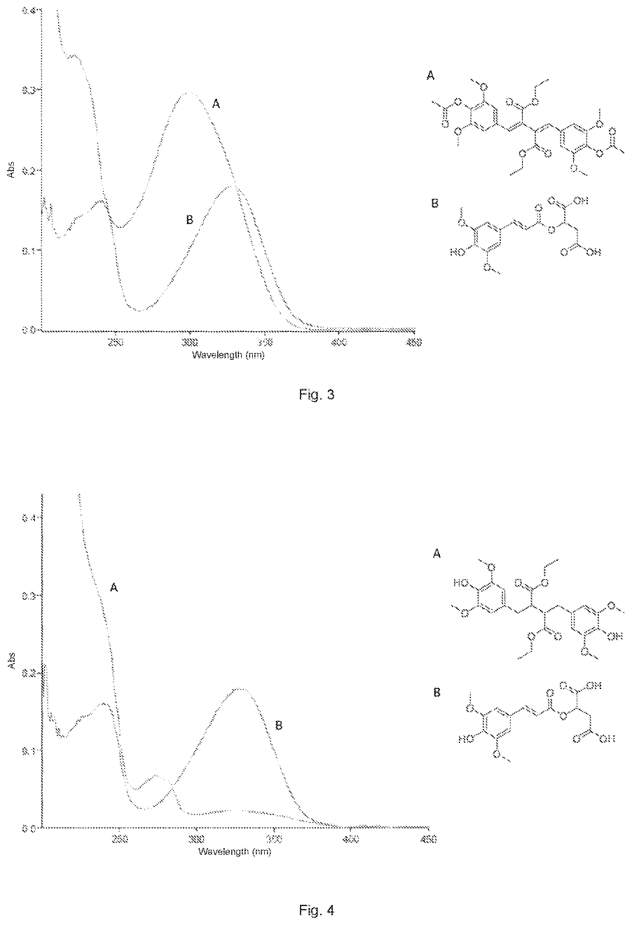

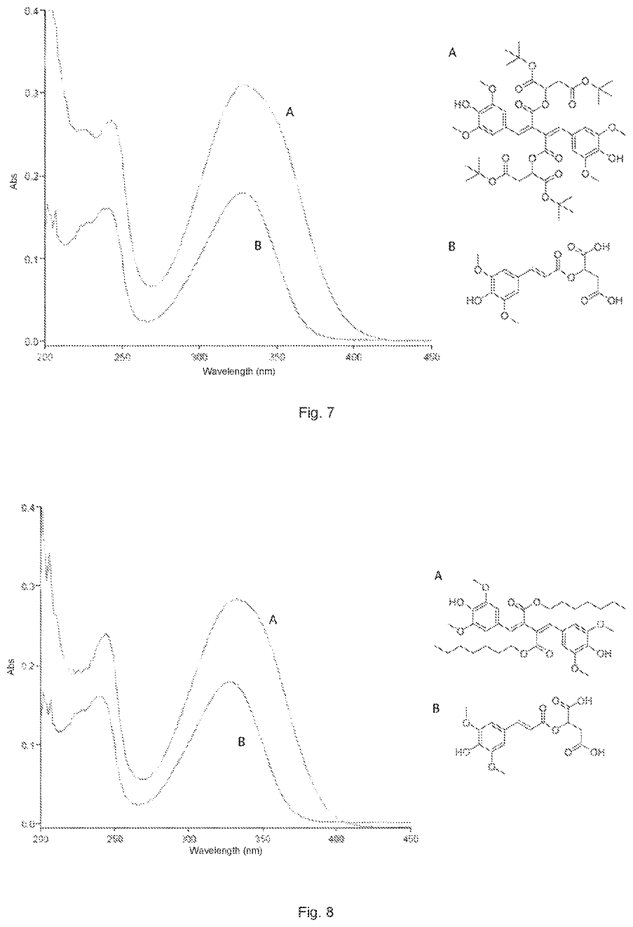

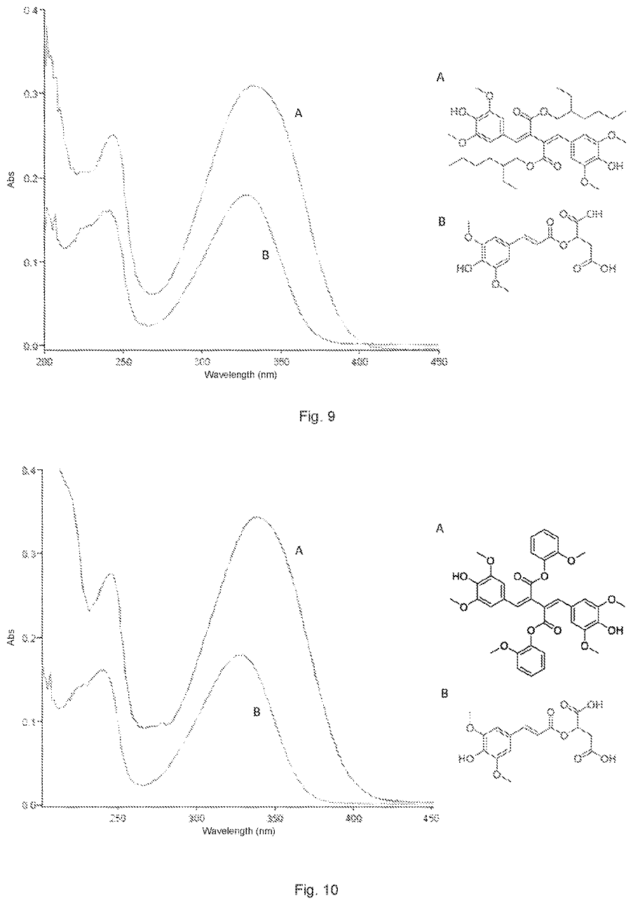

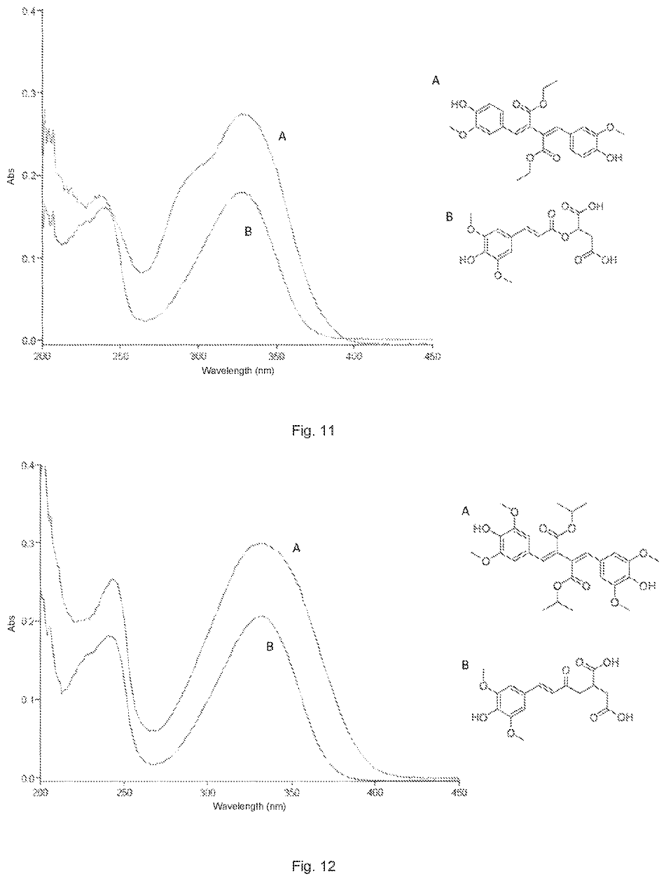

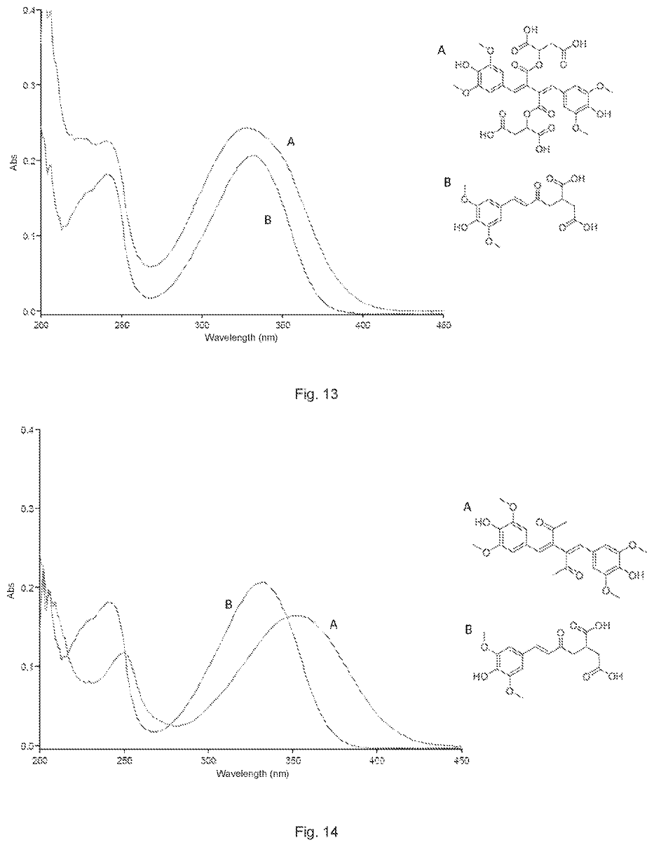

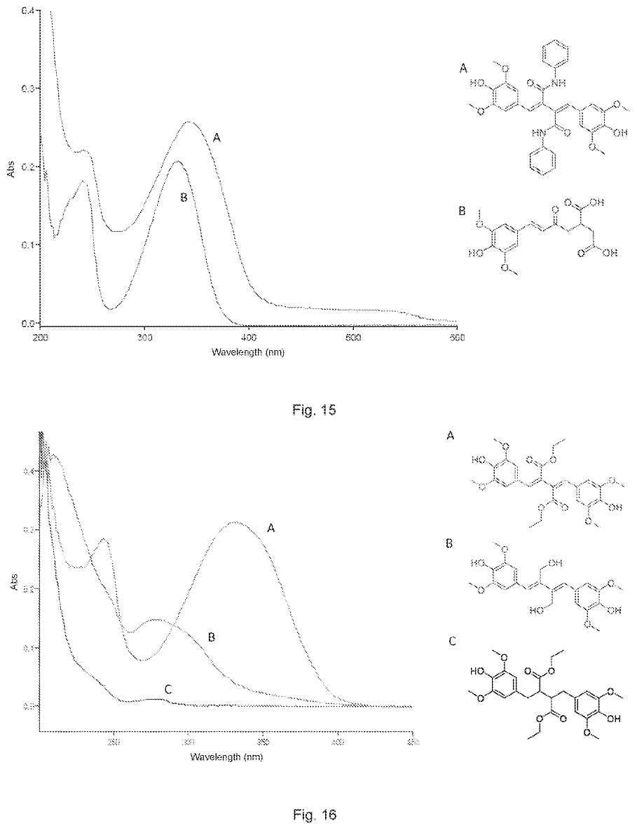

It has been demonstrated that the β-β dimers of formula (I) obtained by the process according to the present disclosure have highly attractive UV absorbance properties, as illustrated in FIGS. 1 to 15. Nevertheless, it has been observed that this anti-UV activity decreases significantly when the compound of formula (I) is reduced to give the compound of formula (III) and is completely absent when the unsaturation of (I) is reduced to give (II) (FIG. 16).

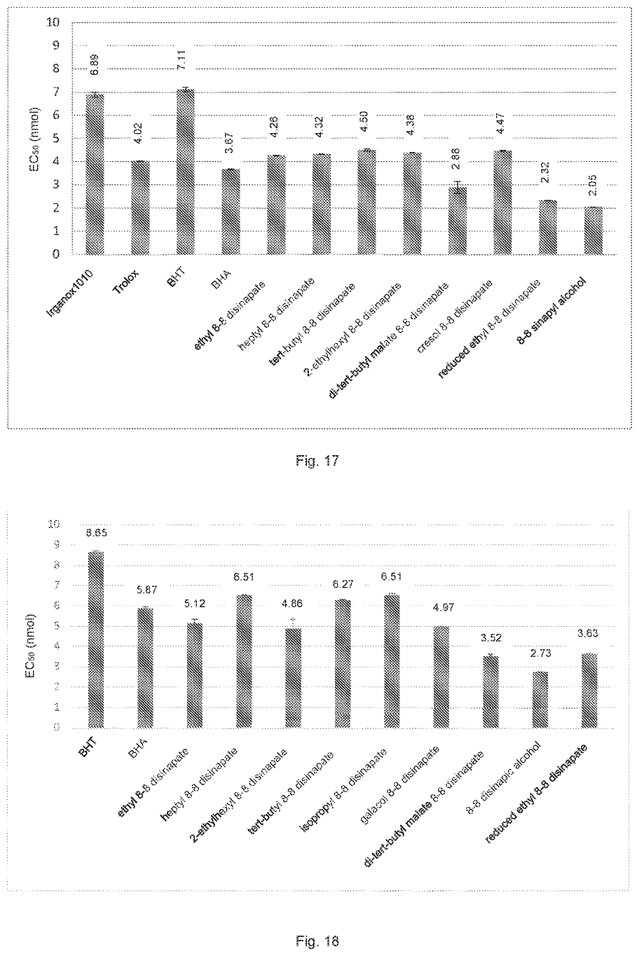

It has also been shown that the nature of the R₆ group modulates the intensity of the absorption and the spectral range of the latter and can confer additional properties. In particular, the β-β dimers of formula (I) where R₆=H have highly beneficial antioxidant properties (FIGS. 17 and 18). It is nevertheless important to note that this antioxidant ability is modulated when the compound of formula (I) is converted into a compound of formula (II) or (III) or (IV).

In a particular embodiment of the present disclosure, the process is carried out from ketones of formula (C). Since ketones are more stable to hydrolysis, they can be used in aqueous solution, for example, for cosmetic applications.

The process according to the present disclosure makes it possible to produce β-β dimers of formula (I), (II), (III) and (IV), the protective properties of which have never before been described. These compounds are particularly beneficial in a large number of applications.

Due to the anti-UV and antioxidant properties, the dimers of formulae (I), (II), (III) and (IV) can be used, in particular, for the production of polymers/plastics (in plastics technology), for the protection of plants against the cold and as cosmetic or food-processing ingredients.

Thus, a second object of the present disclosure relates to novel compounds derived from an ester or an amide of sinapic acid. The present disclosure therefore relates to a compound of formula (I′)

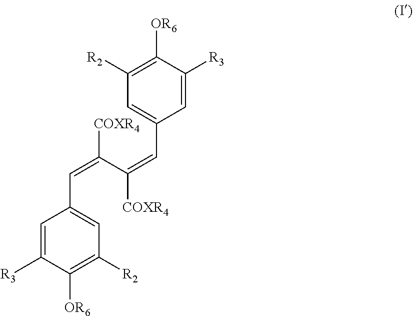

wherein:
   R₂ and R₃ are an OMe group;
   R₄ is a linear, cyclic or branched C₁ to C₃₀ alkyl group or an organic (mono- or di-)acid hydro group;
   R₆ is an H, an acetyl group or a linear, cyclic or branched C₁ to C₈ alkyl group, a silyl, benzyl or benzoyl protecting group;
   X is an O, an NR₅ group wherein R₅ is an H or a linear, cyclic or branched C₁ to C₈ alkyl group;
with the exception of the sinapate ester dimers corresponding to the following CAS numbers: 156257715-4, 1402923-23-2, 1402923-16-3, 1338096-56-2, 1026918-37-5, 915770-86-4, 852713-63-4, 683204-87-7, 389569-68-0, 73119-38-7, 56136-42-6, 53136-39-1, 56136-37-9 and the sinapate amide dimer corresponding to CAS number 88865-57-0.

These compounds can be obtained by the process according to the present disclosure and used in the proposed applications.

A third object of the present disclosure relates to the use of a dimer of formula (I), (II), (III) or (IV) as (i) monomer or additive for preparing an anti-UV and/or antioxidant polymer for the production of plastic (in plastics technology), (ii) molecule for the protection of plants against the cold, (iii) cosmetic ingredient and/or (iv) food-processing additive.

The dimers of formula (I), (II), (III) and (IV) are suitable for the production of plastics that are resistant to UV and to oxidation (in plastics technology). They can be incorporated directly into the plastics during production or applied in the form of a protective surface layer.

Thus, the present disclosure also relates to UV-resistant plastics comprising at least one dimer of formula (I), (II), (III) and/or (IV).

The dimers of formula (I), (II), (III) and (IV) are also useful for the protection of plants against the cold.

The present disclosure therefore also relates to a method for protecting plants against the cold, consisting in applying a composition comprising a dimer of formula (I), (II), (III) and/or (IV) to the plants. Such a composition is preferably in the form of a solution that can be applied, for example, using a spray. In a preferred embodiment of the present disclosure, a composition intended for the protection of plants according to the present disclosure comprises at least one dimer of formula (I), (II), (III) and/or (IV) and another compound that promotes combating cold, such as an antifreeze, a heat reflector, etc.

In the cosmetics field, the dimers of formula (I), (II), (III) and (IV) constitute anti-UV, antioxidant and/or whitening ingredients that can be used for the preparation of creams, powders and perfumes. The possibility of synthesizing these dimers via a green process is an important advantage for cosmetic applications where the absence of toxicity is essential. In a preferred embodiment of the present disclosure, a cosmetic composition according to the present disclosure comprises at least one dimer of formula (I), (II), (III) and/or (IV) and another cosmetic ingredient chosen from a moisturizing agent, an antibacterial agent, an anti-aging agent, etc. Chemical molecules, plant extracts, essential oils, etc., are considered as “cosmetic ingredients.”

The present disclosure therefore relates to a cosmetic composition, such as a cream, a milk, a lotion, a powder, a perfume, comprising dimers of formula (I), (II), (III) and (IV) preferably obtained by the synthesis process according to the present disclosure.

The present disclosure also relates to a food-processing additive comprising a dimer of formula (I), (II), (III) and/or (IV). In a preferred embodiment, the present disclosure relates to a food-processing ingredient comprising at least one dimer of formula (I), (II), (III) and/or (IV) and another food additive such as a preservative, a flavor enhancer, a stabilizer, etc. The present disclosure also relates to a processed food comprising at least one dimer of formula (I), (II), (III) and/or (IV).

### EXPERIMENTAL SECTION

### Example 1: Processes for Synthesizing β-β Dimers

a. Process for preparing the ethyl β-β disinapate dimer using pyridine as reagent and solvent
Ethyl β-β disinapate was prepared from ethyl sinapate and a super-stoichiometric amount of pyridine (C=0.4 M, i.e., approximately 34 equivalents relative to the substrate), which serves both as ligand for the copper and as solvent. Copper bromide was used as the reaction catalyst (0.1 eq.). The reaction was carried out by heating at 50° C. for 24 hours in the presence of air or oxygen. The conversion and the yield obtained are, respectively, 96% and 62% after purification on a silica column.

b. Preparation of the other β-β dimers from the ethyl β-β disinapate
Conventional hydrogenation using palladium on carbon was carried out to obtain a saturated compound of ethyl didihydrosinapate type.
A reduction of the esters to alcohol was carried out to obtain the β-β dimer of disinapyl alcohol.

c. Process for preparing the ethyl β-β disinapate dimer using pyridine as reagent only and a polar aprotic solvent (DMSO in this case)
Ethyl β-β disinapate was prepared from ethyl sinapate (1 eq.) and a stoichiometric amount of pyridine (1 equivalent relative to the substrate), copper bromide (0.1 eq.) in DMSO (C=0.5 M/substrate). The reaction was carried out by heating at 50° C. for 20 hours in the presence of air or oxygen. The conversion and the yield obtained are, respectively, 100% and 60% after purification on a silica column.

d. Process for preparing the ethyl β-β disinapate dimer using pyridine as reagent and 2H-LGO as solvent.
Ethyl β-β disinapate was prepared from ethyl sinapate (1 eq.) and a sub-stoichiometric amount of pyridine (0.76 equivalents relative to the substrate), copper bromide (0.1 eq.) in 2H-LGO (C=1.8 M/substrate). The reaction was carried out by heating at 51.5° C. for 7 hours in the presence of air or oxygen. The conversion and the yield obtained are, respectively, 100% and 89% after ethyl acetate/1 M aqueous HCl extraction and without any further purification step.

TABLE 1
Summary of the conditions used for the synthesis of the ethyl β-β
disinapate dimer (ethyl sinapate 1 eq., CuBr (0.1 eq.)) based on the solvent used

| Pyridine | Solvent (M/ethyl | Temperature | Reaction | Conversion | Yield |
|---|---|---|---|---|---|
| (eq.) | sinapate) | (° C.) | time (h) | (%) | (%) |
| 31 | Pyridine (0.4) | 50 | 24 | 96 | 62 |
| 1 | DMSO (0.5) | 50 | 20 | 100 | 60 |
| 0.76 | 2H-LGO (1.8) | 51.5 | 7 | 100 | 89 |

### Example 2: Properties of the β-β Dimers

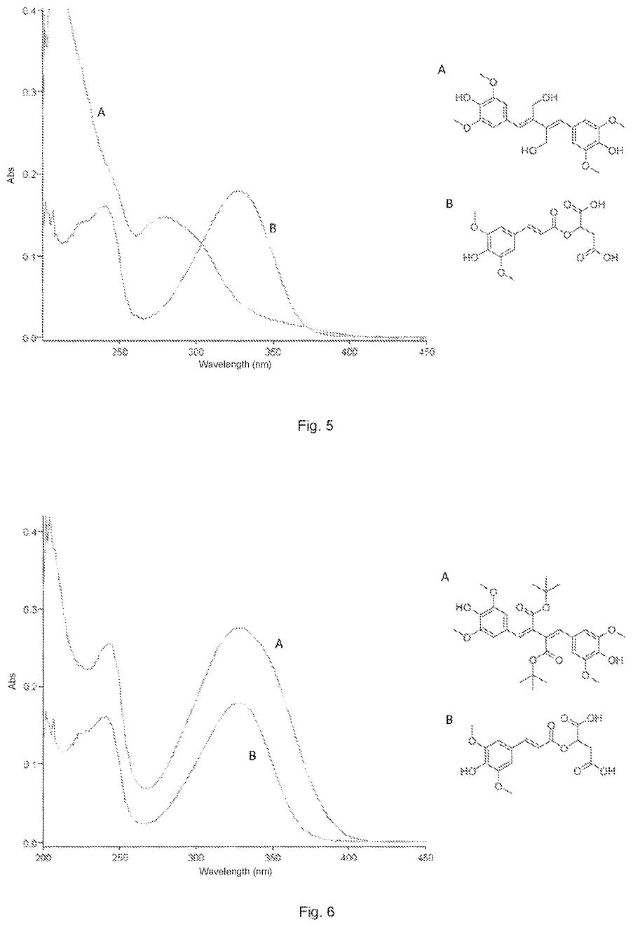

a. UV absorbance
To carry out this experiment, a solution of the compound to be studied at a concentration of 10 μmol·L⁻¹ in ethanol was produced.
The absorbance properties of different dimers were measured in comparison with those of sinapoyl malate, a molecule with known high anti-UV properties.
The results confirmed the need for extended conjugation and unexpectedly showed that the ethyl β-β disinapate dimer (A) proves to have higher anti-UV properties than sinapoyl malate (B) since the absorption band of this dimer is at 200-400 nm, compared to 200-370 nm for sinapoyl malate (FIG. 1).
FIGS. 1 to 16 illustrate the results obtained. These results show, in particular, that ethyl disinapate, as well as certain derivatives, have beneficial anti-UV properties since they are superior to that of sinapoyl malate (FIGS. 1, 2, 3, 6, 7, 8, 9, 10, 11, 12, 13 and 15). On the other hand, when the compound is saturated by hydrogenation, it loses these properties (FIG. 4), just as when the esters are reduced to give alcohol (FIG. 5); this result is also presented in FIG. 16. A change of the R₃ radical can also affect these properties (FIG. 11).

b. Antioxidant Properties
The antioxidant activity of the synthesized dimers having free phenols (R₆=H) was analyzed by the ABTS method (the lower the EC₅₀, the greater the antioxidant ability) then compared with that of IRGANOX® 1010, trolox, BHT and BHA, commonly used commercial antioxidants.
The ester dimers exhibit extremely beneficial anti-free radical properties, all of which are superior to that of BHT and of the same order as, or even superior to, that of BHA (FIGS. 17 and 18).
Dimers with reduced conjugation (β-β disinapyl alcohol and reduced ethyl β-β disinapate) exhibit even stronger properties.
In conclusion, the results described in this document demonstrate the potential of the dimers of formula (I), (II), (III) and (IV) for use as anti-UV and/or antioxidant, possessing the advantage of being obtained from a compound of natural origin, via a sustainable process and without a purification step when the solvent used is predominantly 2H-LGO. If both the anti-UV and antioxidant properties of the dimers synthesized are considered, it appears that ethyl β-β disinapate is a particularly beneficial compound.

1. A process for synthesizing a dimer of formula (I): 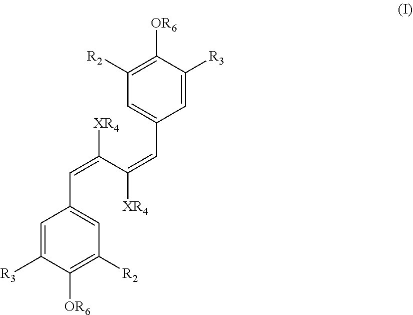
wherein:
R₂ and R₃ are independently selected from a linear, cyclic or branched C₁ to C₈ alkyl group or a linear, cyclic or branched C₁ to C₈ O-alkyl group:
R₄ is an H, a linear, cyclic or branched C₁ to C₃₀ alkyl group, or an organic (mono- or di-)acid hydro group:
R₆ is an H, an acetyl group or a linear, cyclic or branched C₁ to C₈ alkyl group, a silyl, benzyl or benzoyl protecting group:
X is a C═O group, a CO₂ group or a C(O)NR₅ group, wherein R₅ is an H or a linear, cyclic or branched C₁ to C₈ alkyl group;
from esters or amides of a para-hydroxycinnamic acid disubstituted in the ortho position with respect to the phenol function (such as sinapic acid) when X is a CO₂ or C(O)NR₅ group, or from ketones of formula (C): 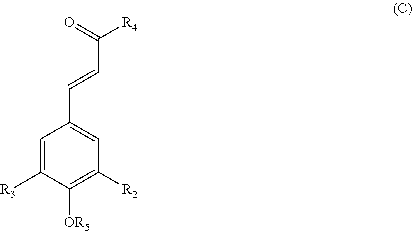
when X is a C═O group,
wherein the process implements a catalytic reaction with copper in the presence of an amine, alone or in combination with a polar aprotic solvent.

2. The process of claim 1, wherein the catalytic reaction is carried out in the presence of an amine and a polar aprotic solvent and wherein the amount of amine is reduced such that the amine acts only as a catalytic reagent.

3. The process of claim 2, wherein the amine is chosen from pyridine, 4-dimethylaminopyridine, piperidine, piperazine, trimethylamine, pyrrolidine and aniline.

4. The process of claim 3, wherein the polar aprotic solvent is chosen from N-methyl-2-pyrrolidone, dimethylformamide, dimethyl sulfoxide, 1,4-dioxane, tetrahydrofuran, dichloromethane and dihydrolevoglucosenone.

5. The process of claim 2, wherein the amine is pyridine or DMAP and the solvent is dihydrolevoglucosenone.

6. The process of claim 4, wherein the dimer of formula (I) is ethyl β-β disinapate or β-β disinapoyl malate.

7. A process for preparing a dimer of formula (II), (III) or (IV): 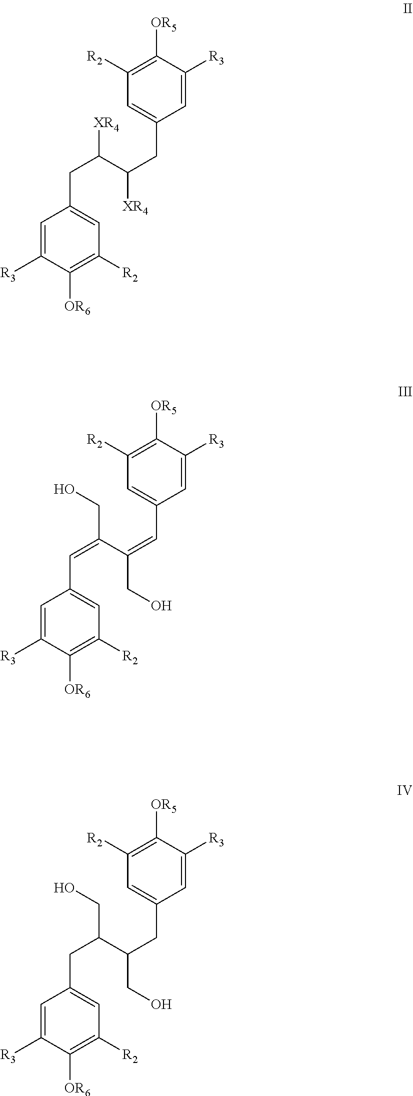
obtained by reduction of dimer of formula (I): 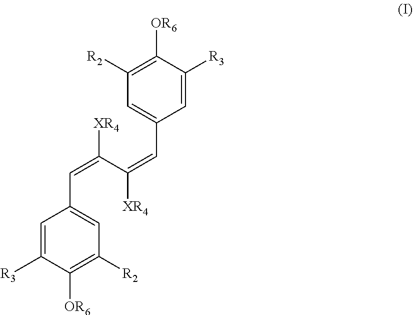
wherein:
R₂ and R₃ are independently selected from a linear, cyclic or branched C₁ to C₈ alkyl group or a linear, cyclic or branched C₁ to C₈ O-alkyl group:
R₄ is an H, a linear, cyclic or branched C₁ to C₃₀ alkyl group, or an organic (mono- or di-)acid hydro group;
R₆ is an H, an acetyl group or a linear, cyclic or branched C₁ to C₈ alkyl group, a silyl, benzyl or benzoyl protecting group;
X is a C═O group, a CO₂ group or a C(O)NR₅ group, wherein R₅ is an H or a linear, cyclic or branched C₁ to C₈ alkyl group.

8. A compound of formula (I′) 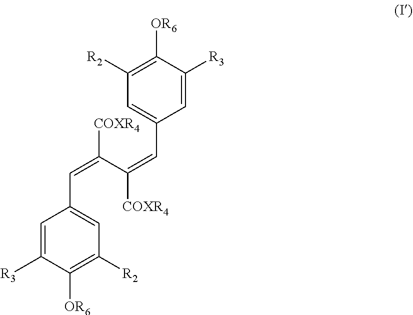
wherein:
R₂ and R₃ are an OMe group;
R₄ is a linear, cyclic or branched C₁ to C₃₀ alkyl group or an organic (mono- or di-)acid hydro group;
R₆ is an H, an acetyl group or a linear, cyclic or branched C₁ to C₈ alkyl group, a silyl, benzyl or benzoyl protecting group;
X is an O, an NR₅ group wherein R₅ is an H or a linear, cyclic or branched C₁ to C₈ alkyl group;
with the exception of the sinapate ester dimers corresponding to the following CAS numbers: 156257715-4, 1402923-23-2, 1402923-16-3, 1338096-56-2, 1026918-37-5, 915770-86-4, 852713-63-4, 683204-87-7, 389569-68-0, 73119-38-7, 56136-42-6, 53136-39-1, 56136-37-9 and the sinapate amide dimer corresponding to CAS number 88865-57-0.

9.-14. (canceled)

15. The process of claim 1, wherein the amine is chosen from pyridine, 4-dimethylaminopyridine, piperidine, piperazine, trimethylamine, pyrrolidine and aniline.

16. The process of claim 1, wherein the polar aprotic solvent is chosen from N-methyl-2-pyrrolidone, dimethylformamide, dimethyl sulfoxide, 1,4-dioxane, tetrahydrofuran, dichloromethane and dihydrolevoglucosenone.

17. The process of claim 1, wherein the amine is pyridine or DMAP and the solvent is dihydrolevoglucosenone.

18. The process of claim 1, wherein the dimer of formula (I) is ethyl β-β disinapate or β-β disinapoyl malate.
